(19) United States
(12) Patent Application Publication
(10) Pub. No.: US 2020/0170888 A1
(43) Pub. Date: Jun. 4, 2020

(54) SMART SYSTEM FOR DISPENSING MEDICATIONS IN REAL-TIME AND METHODS EMPLOYED THEREOF

(71) Applicant: KAMANA MISRA, Newtown, PA (US)

(72) Inventor: KAMANA MISRA, Newtown, PA (US)

(21) Appl. No.: 16/711,614

(22) Filed: Dec. 12, 2019

Related U.S. Application Data
(60) Provisional application No. 62/774,537, filed on Dec. 3, 2018.

(51) Int. Cl. A61J 7/04; G16H 10/60; G16H 20/13; A61J 7/00

(52) U.S. Cl. CPC A61J 7/049; G16H 10/60; G16H 20/13; A61J 2200/30; A61J 7/0427; A61J 2205/60; A61J 7/0069

(57) ABSTRACT

Exemplary embodiments of the present disclosure are directed towards a smart system and method for generating medication alerts and communicating to a medication dispensing device in real-time. The smart system comprises a plurality of computing devices in wireless communication with at least one medication dispensing device, the plurality of computing devices comprises of a medication management module configured to generate schedules and timetables based on a plurality of inputs provided by a plurality of users with reference to the medication intended to be housed in the at least one medication dispensing device based on the consumption requirement, the medication management module configured to send the generated schedules and timetables to the at least one medication dispensing device. The smart system further comprises cloud servers configured to establish communication with the plurality of computing devices and the at least one medication dispensing device via a network, the cloud servers are configured to store the conversations between the plurality of computing devices and the at least one medication dispensing device.

### CROSS-REFERENCE TO RELATED APPLICATION

This application claims the benefit of U.S. Provisional Application No. 62/774,537, filed Dec. 12, 2018.

### TECHNICAL FIELD

The present disclosure generally relates to the field of automated pill dispensing systems. More particularly, the present disclosure relates to a system and method for generating medication alerts and communicating to a medication dispensing device in real-time based on the drug to drug interactions and clinical pharmacology.

### BACKGROUND

Quest for the improvement of quality of human life has led to tremendous improvement in medical science. These improvements range from sophisticated equipment to medication requirements. From the medication perspective, conditions must be ensured for appropriate dispensing of pills, at an appropriate time interval, and to the right user. Pills are oral dosage formulations preferably as tablets or capsules. Medications which need to be taken on a regular basis and at prescribed intervals need judicious management of time. Managing a dosage regimen can be frustrating and confusing. Physicians prescribe multiple medications for different health conditions. Patients undergoing treatment for multiple disorders may end up forgetting or jumbling their doses. Some medications are to be consumed once a day or twice a day or thrice a day, whereas some may be at weekly intervals.

Patients do not understand the properties of drugs and combining multiple medicines and taking them at the same time could produce adverse effects biologically. Pharmacodynamics and pharmacokinetics need to be considered before taking medications and this requires an automated pharmacovigilance system that will control the pill dispenser remotely based on the drug interactions which are personalized for every user.

Plurality of systems has been used earlier to maintain over and under medication. The complexities involved post consumption of drug whether more or less cannot be understood by every patient. Thus medication compliance coupled with patient's safety is needed.

### BRIEF SUMMARY

The following presents a simplified summary of the disclosure in order to provide a basic understanding to the reader. This summary is not an extensive overview of the disclosure and it does not identify key/critical elements of the invention or delineate the scope of the invention. Its sole purpose is to present some concepts disclosed herein in a simplified form as a prelude to the more detailed description that is presented later.

Exemplary embodiments of the present disclosure are directed towards a smart system for dispensing medications in real time and methods employed thereof.

An exemplary object of the present disclosure is directed towards monitoring the medication time and alerts.

Another exemplary object is directed towards automated access to the pills.

Another exemplary object is directed towards generation of patient friendly alerts.

Another exemplary object is directed towards auto generation of time table and/or schedule.

Yet another exemplary object of the present disclosure is directed towards auto generation of reminders.

An exemplary aspect, a smart system for dispensing medications comprising a plurality of computing devices in wireless communication with at least one medication dispensing device, the plurality of computing devices comprises of a medication management module configured to generate schedules and timetables based on a plurality of inputs provided by a plurality of users with reference to the medication intended to be housed in the at least one medication dispensing device based on the consumption requirement.

According to another exemplary aspect, the medication management module configured to send the generated schedules and timetables to the at least one medication dispensing device.

According to another exemplary aspect, the smart system further comprises cloud servers are configured to establish communication with the plurality of computing devices and the at least one medication dispensing device via a network, the cloud servers are configured to store the conversations between the plurality of computing devices and the at least one medication dispensing device.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a block diagram depicting an environment for dispensing time bound medications, according to an exemplary embodiment of the present disclosure.

FIG. 2A is a block diagram depicting the medication management module 112 shown in FIG. 1, in accordance with one or more exemplary embodiments.

FIG. 2B is a diagram depicting an example screen that may appear on a visual display of a computing device 102a or 102b, in accordance with one or more exemplary embodiments.

FIG. 3 is a block diagram depicting the medication dispensing device 104 as shown in FIG. 1, according to an exemplary embodiment of the present disclosure.

FIG. 4 is an example diagram depicting the cross section view of the medication dispensing device 104, according to an exemplary embodiment of the present disclosure.

FIG. 5 is an example diagram depicting the side view of the medication dispensing device 104, according to an exemplary embodiment of the present disclosure.

FIG. 6 is an example diagram depicting the top view of the tray with compartments, according to an exemplary embodiment of the present disclosure.

FIG. 7 is a flowchart depicting the method for dispensing time bound medications, according to an exemplary embodiment of the present disclosure.

FIG. 8 is a flowchart depicting the method for performing actions between the medication dispensing device and the computing device, according to an exemplary embodiment of the present disclosure.

FIG. 9 is a flowchart depicting the method for managing actions by the medication management module, according to an exemplary embodiment of the present disclosure.

FIG. 10 is a block diagram depicting the details of the computing device in which various aspects of the present disclosure are operative by execution of appropriate software instructions.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

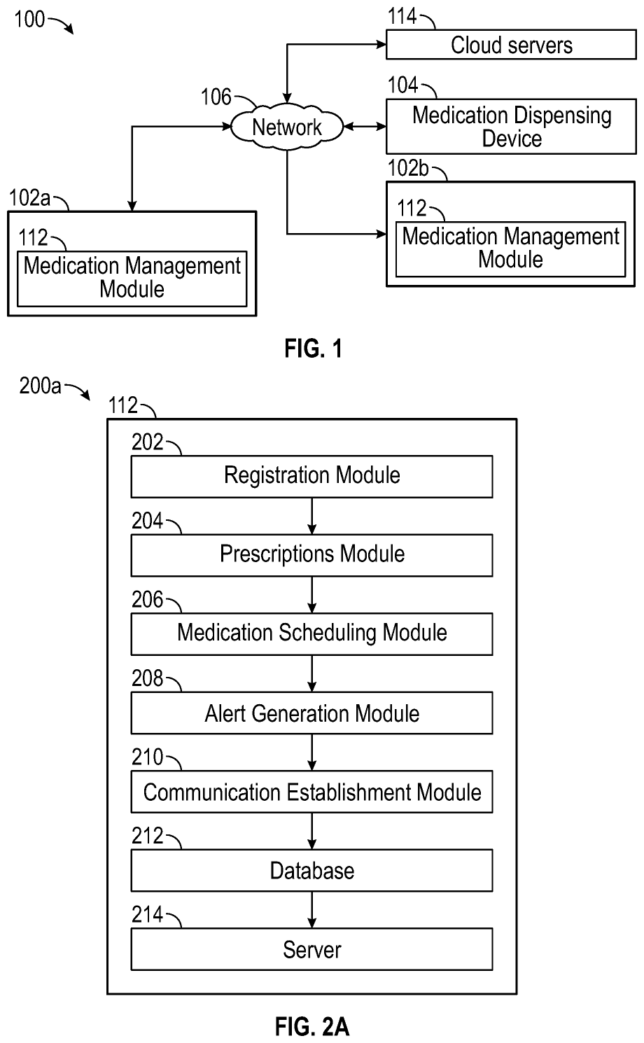

Referring to FIG. 1 is a block diagram 100, depicting an environment for dispensing time bound medications, according to an exemplary embodiment of the present disclosure. The environment 100 includes a first computing device 102a, a second computing device 102b, a network 106, a medication dispensing device 104, and cloud servers 114. Computing devices 102a-102b are used herein to refer to any device with processing capability such that it can execute instructions. Those skilled in the art will realize that such processing capabilities are incorporated into many different devices and therefore the term computing devices 102a-102b include web servers, smartphones, PCs, servers, tablet computers, personal digital assistants, watch, and many other electronic computing devices. The network 106 may comprise of, not limited to, Ethernet, a wireless local area network (WLAN), or a wide area network (WAN), a ZigBee wireless network, a Bluetooth wireless network (short-range communication), a WIFI, Lorawan, Sigfox, LPWAN, GSM/GPRS/LTE wireless communication networks, HTTPS, TCP/IP (long range-wireless communication) and the like. The first computing device 102a may only be operated by a first user. Here, the first user may include but not limited to, a clinician, a doctor, a nurse, a patient, a relative of the patient, a friend, an employee, and the like. The second computing device 102b may only be operated by a second user. The second user may include but not limited to, a physician, a paramedic, a nurse, a caregiver, and the like. The first computing device 102a will be loaded with software/hardware/firmware pharmacovigilance module that will automatically compute based on the pre-loaded instructions. The medication dispensing device 104 may not be limited to a pillbox, which is a controlling and communication unit and trays with multiple compartments, where tray may be inserted for storing the prescribed medication by the user. In preferred embodiments, a tray may be inserted in any available slot. As soon as the tray is inserted in the slot, the medication dispensing device 104 automatically identifies the medications stored in the tray or fed through network 106 from medication management module 112 through software instructions and the tray is automatically locked into the slot. The term “module” is used broadly herein and refers generally to a software or hardware or firmware program resident in memory of the computing devices 102a-102b. The medication may not be limited to multiple time slots through the day, week, and a month. The computing devices 102a-102b comprise of a medication management module 112 configured to generate a schedule and/or timetable based on the inputs provided by the user with reference to the medication intended to be housed in the medication dispensing device 104 based on the consumption requirement. The medication management module 112 may be accessed as not limited to, a mobile application, a web application, a portal, a voice-enabled application and/or other software application that offers the functionality of accessing mobile applications, and viewing/processing of interactive pages, for example, are implemented in the computing devices 102a or 102b as will be apparent to one skilled in the relevant arts by reading the disclosure provided herein. The cloud servers 114 are configured to establish communication with the computing devices 102a-102b and the medication dispensing device 104 via the network 106. The cloud servers 114 are configured to store the conversations between the computing device 102a or 102b and the medication management module 112.

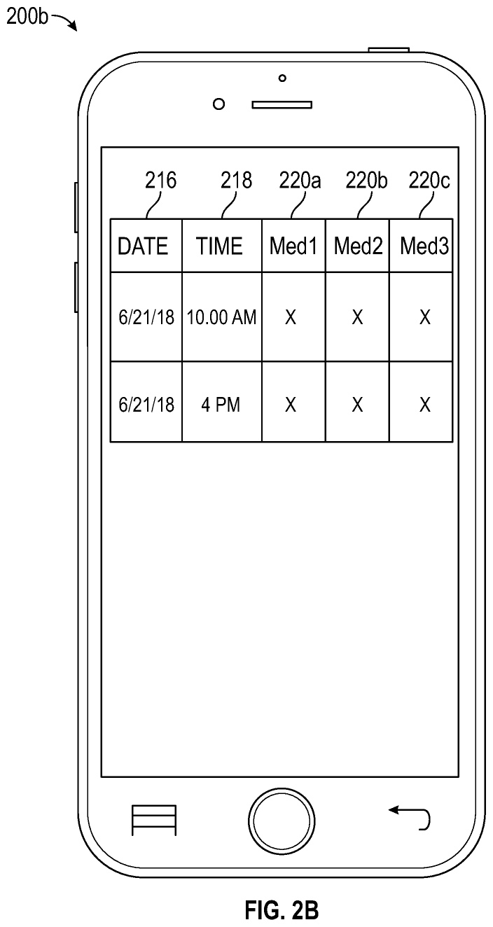

Referring to FIG. 2A is a block diagram 200, depicting the medication management module 112 shown in FIG. 1, in accordance with one or more exemplary embodiments. The medication management module 112 is executed by a processor of the computing device 102a or 102b. The graphical interfaces are displayed on a display monitor (generally screens made of led, OLED or LCD, and like) of the computing device 102a or 102b when the medication management module 112 is executed. The users allow accessing the medication management module 112 by entering login identity credentials. The users may include but not be limited to, the first users, the second users, and the like. The credentials may include a unique identifier or identifiers of the medication management module 112. For example, identifiers may include a username, an email address, an account identity, a mobile number, finger print, retina check or NFC based user authentication from mobile and the like. A secured code associated with an identifier may include a password, a symmetric encryption key, biometric values, a passphrase, and the like. The medication management module 112 comprises a registration module 202, a prescriptions module 204, a medication scheduling module 206, an alert generation module 208, a communication establishment module 210, a database 212, and a server 214. Upon accessing an account on the medication management module 112 by the user and providing medication details in the computing device 104, medication timetable is generated by the database 212 and the generated timetable is transmitted to the computing device 102a or 102b and the medication dispensing device 104. The database 212 may also be alternatively referred to as a pharmacovigilance database which organizes, manages, and updates the information uploaded regarding the medication schedule as uploaded by the user.

The registration module 202 facilitates the setting up of a user's account in the application and/or web portal which is utilized to enter the details of prescribed medication. The prescriptions module 204 is configured to acquire and store prescription information from, for example, personal health records of the users, health-related information, and the like. The prescription information is provided by the second users. The medication scheduling module 206 is configured to generate medication schedules for each of the user. The medication schedules are based on the acquired prescriptions. The medication schedules may not be limited to multiple time slots through the day, week, and a month. The alert generation module 208 is configured to generate reminders and alerts for the users based on the generated medication schedules. The reminders and alerts may contain one or more predefined audio and/or text messages intended to alert the user about the discrepancy in medication dispensing. The communication establishment module 210 is configured to establish communication with the medication dispensing device 104 and also configured to establish communication with other computing devices. The database 212 is configured to generate the real-time alerts to the computing device 102a or 102b, thus initiating the conversation with the medication dispensing device 104 based on the generated prescribed medication timetable. The server 214 processes the inputs by the user from one's computing device 102a or 102b. The display unit of the computing device 102a or 102b aids the user to enter the details of the prescribed medication manually. Also, upon the consumption of the prescribed medication taken from one of the trays of the medication dispensing device 104 a notification is sent to the server 214. The database 212 stores the record of the user of having consumed the prescribed medication which may not be limited to a tablet, capsule, a pill, and the like.

Referring to FIG. 2B is diagram 200b, depicting an example screen that may appear on a visual display of the computing device 102a or 102b, in accordance with one or more exemplary embodiments. The example screen 200b shows a date option 216, a time option 218, medication options 220a, 220b, and 220c. The date option 216 is configured to provide options which are daily, weekly, monthly, and the like. The date option 216 is further configured to enable the users to select the date. The time option 218 shows the scheduled administration time in hours and minutes, the scheduled administration time may also include pre- and post-prandial times, bedtime, as well as optional before and after modifiers if the medication is to be taken before or after a meal, rather than during, and the like. The medication options 220a, 220b, and 220c include information about each of the medications that are listed in the daily dosage schedule, weekly dosage schedule, monthly dosage schedule, and the like. The information listed for each medication may include but is not limited to, the medication name, medication pictures, the active ingredient/generic name and/or trade name, a medication class, an indication for the medication, uses, and/or warnings.

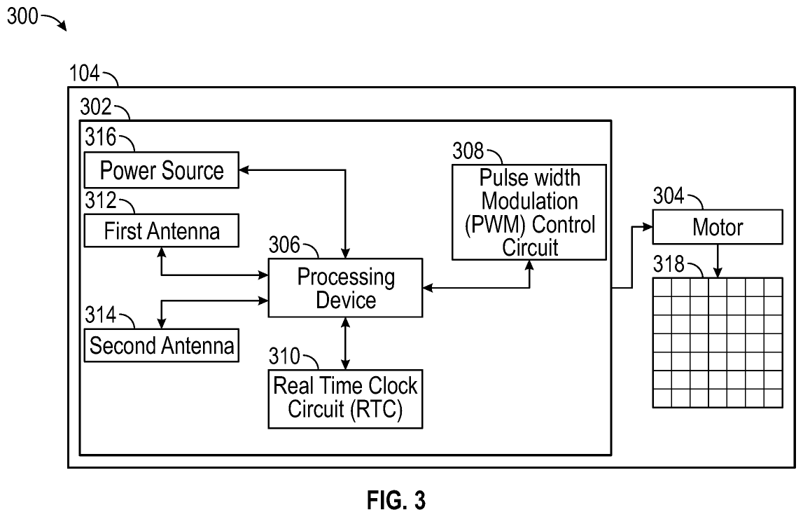

Referring to FIG. 3 is a block diagram 300, depicting the medication dispensing device 104 as shown in FIG. 1, according to an exemplary embodiment of the present disclosure. The medication dispensing device 104 consisting of a controlling and communication unit 302. The controlling and communication unit 302 comprises of a processing device 306, a pulse width modulation (PWM) control circuit 308, a real time clock circuit (RTC) 310, a first antenna 312, a second antenna 314 and a power source 316. The medication dispensing device 104 further comprises trays with compartments 318, where each compartment is dedicated for the dosage of the medications. The trays with compartments 318 opens and/or closes with the aid of a motor 304, not limited to a DC motor. The motor 304 serves as the dispensing control mechanism. The maintenance of time is critical in cases of accurate medical dispensing requirements. The real time clock circuit (RTC) 310 comprises a crystal and a date and time clock based RTC. The power source 316 could transmit power from a standby battery or through direct current connected to a power source. The action related to schedule i.e. date and time takes place in the RTC circuit 310 which is connected to the processing device 306 which generates the schedule for each unit of the prescribed medication in each tray of the medication dispensing device 104. The processing device 306 may include but is not limited to, a microcontroller (for example ARM 7 or ARM 11), a raspberry pi, a microprocessor, a digital signal processor, a microcomputer, a field programmable gate array, a programmable logic device, a state machine or a logic circuitry. The processing device 306 also read the date and time data from the RTC circuit 310. The RTC circuit 310 could be internal to the processing device 306 or it could be an external circuit with respect to the processing device 306. The RTC circuit 310 may run on the DC/AC power unless no power is provided, in which case it may run on the battery.

In accordance with one or more exemplary embodiments of the present disclosure, the first antenna 312 and the second antenna 314 are coupled to the processing device 306. The first antenna 312 and the second antenna 314 are configured to establish communication with the computing device 102a or 102b using dedicated wireless communication technologies via the network 106. For e.g., near field communication (NFC) or radio frequency identification (RFID), that are entirely distinct from the main wireless communication technology (Wi-Fi, for e.g.). In some implementations, the first antenna 312 may be a long-range communication antenna, whereas the second antenna 314 may be a short-range communication antenna. In other implementations, however, a single antenna (first antenna 312 or second antenna 314) may be used. In an alternative embodiment the functionalities of first and second can be combine into one single antenna. The medication dispensing device 104 can interface with the computing device 102a or 102b over long range and wireless networks and/or short range wireless networks via the first antenna 312 and the second antenna 314. The power source 316 is configured to supply electric power to not limiting to the controller which is a device that supplies electrical energy to one or more electric loads, for example, a battery.

When the medication dispensing device 104 receives communication from the computing device 102a or 102b, a signal is transmitted to the PWM control circuit 308 from the processing device 306, thus activating a motor 304 (DC) to either push or pull the trays with compartments 318 by the PWM control circuit 308. In an embodiment, the processing device 306 having embedded software configured for turning the motor 304 in a particular direction for a predetermined set of time (4 seconds, for e.g.). The motor 304 speed is controlled using the PWM control circuit 308. In another embodiment the rotations can be controlled through the encoder circuit inside the DC motor. Once the medication dispensing device 104 is activated, a communication is transmitted to the computing device 102a or 102b followed by storing the activity record at the database 212. Once the prescribed medicine is taken from the medication dispensing device 104, an RF notification appears on the computing device 102a or 102b, thus transmitting a notification to the server 214 from the computing device 102a or 102b and storing the final communication in the backend server 214 that the user consumed the prescribed medicine. The medication dispensing device 104 may further comprise LEDs configured to indicate the time for medicine. The medication dispensing device 104 dispenses the medication. However, this only happens when it is time for the user (e.g., patient) to take the medication. In order to enable this procedure, the medication dispensing device 104 is equipped with a touch sensor or finger print sensor. The touch/finger print sensor is electrically coupled to the processing device 306. The medication dispensing device 104 sends a message to the computing device 102a or 102b once the touch/finger print sensor is activated, and the database 212 may store the activity record.

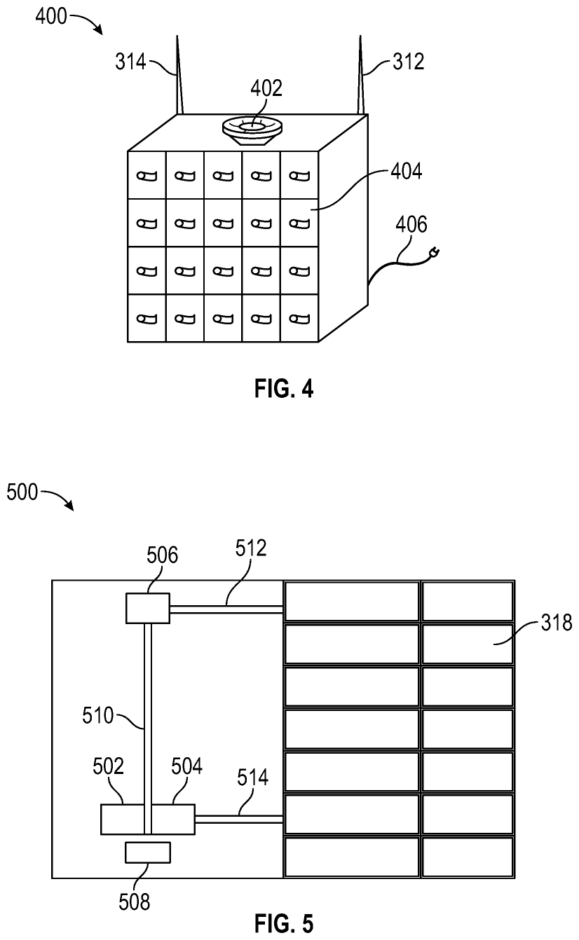

Referring to FIG. 4 is an example diagram 400, depicting the cross section view of the medication dispensing device 104, according to an exemplary embodiment of the present disclosure. The cross section view 400 depicting the first antenna 312, the second antenna 314, a speaker 402, a knob 404, and a power line 406. The speaker 402 is configured for playing live/recorded messages. The speaker 402 that may alert the users when it is time to take a medication or when the medication has not been taken. The knob 404 is positioned on top of each tray with compartments 318 and it rotates about a common rotation axis. For example, the user can manually rotate the knob 404 to open the tray with compartments 318. The power line 406 that can be plugged into an external power supply.

Referring to FIG. 5 is an example diagram 500, depicting the side view of the medication dispensing device 104, according to an exemplary embodiment of the present disclosure, the side view 500 depicting a first motor 502, a second motor 504, a third motor 506, a circuitry 508, a first robotic arm 510, a second robotic arm 512, a third robotic arm 514, and the trays with compartments 318. The first motor 502 is configured to shift the first robotic arm 510 from left to right and vice versa. The second motor 504 is configured to shift the second robotic arm 512 from up to down and vice versa. The third motor 506 is configured to shift the third robotic arm 514 from up to down and vice versa. The all the three robotic arms 510, 512, 514 are configured to push or pull the trays 318. The circuitry 508 is connected to the first motor 502, the second motor 504, and the third motor 506 and allowing the motors 502, 504, 506, to be rotated in a particular direction.

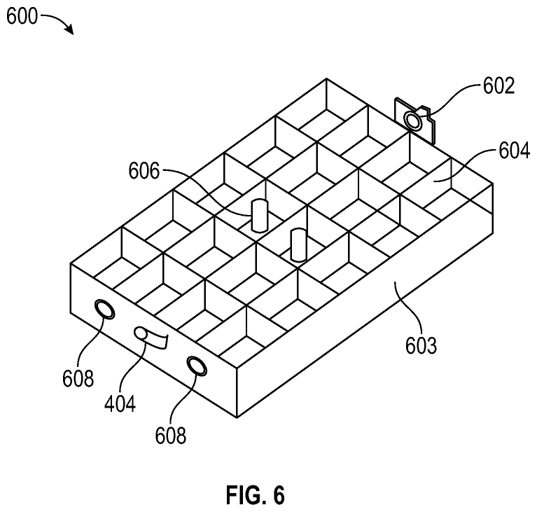

Referring to FIG. 6 is an example diagram 600, depicting the top view of the tray with compartments, according to an exemplary embodiment of the present disclosure. The top view 600 depicting a camera 602, a tray 603, compartments 604, pills 606, touch sensors 608, and the knob 404. The camera 602 is configured to detect at least one of the open or closed state of the tray 603, the presence or absence or identification of the pills 606 in the compartments 604. The user has an access to the pills 606 and opening of the tray 603 post activation of the touch sensors 608. The opened tray 603 finally reverts to its position post the deactivation of the touch sensors 608.

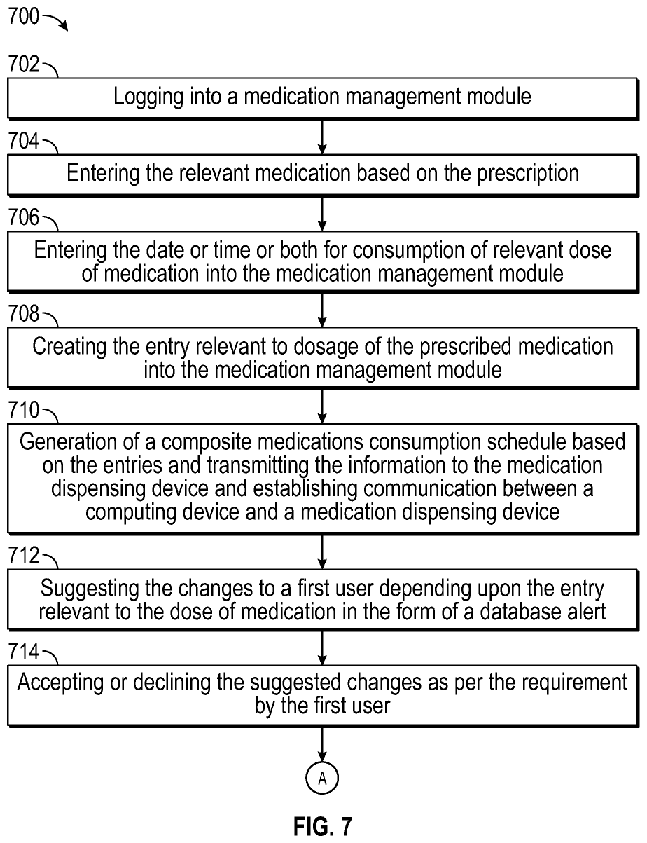

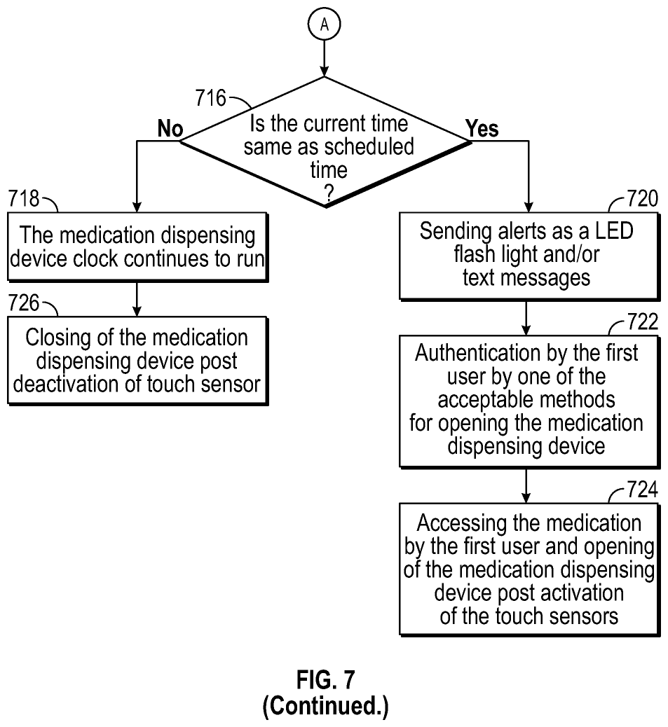

Referring to FIG. 7 is a flowchart 700, depicting the method for dispensing time bound medications, according to an exemplary embodiment of the present disclosure. As an option, the method 700 may be carried out in the context of the details of FIG. 1, FIG. 2, FIG. 3, FIG. 4, FIG. 5, and FIG. 6. However, the method 700 may also be carried out in any desired environment. Further, the aforementioned definitions may equally apply to the description below.

The method commences at step 702 where the user sets up an account in the medication management module. The information input may not be limited to a username and other statistics vital to the user's health if one is the consumer of the prescribed medication. The detail of relevant medication which has been prescribed by the second user to the first user of the prescribed medication is entered as details in the medication management module at step 704. The details may not be limited to, name of the health condition, the name of the prescribed medication not limiting to, a brand name and/or a generic name of the prescribed medication intended to be placed in the medication dispensing device's tray. The details of date and time prescribed for consumption of the prescribed medication is entered by the user at step 706. The relevant dosage for the prescribed medication is also fed as a further input at step 708. Generation of a composite medication/s consumption schedule based on the entries and transmitting the information to the medication dispensing device and establishing communication between the computing device and the medication dispensing device takes place at step 710. Changes are suggested to the user (preferably patient) and are reflected as a database alert to the user at step 712. These changes are dependent on the entry done by the user relevant to the dosage and output from pharmacovigilance database. At step 714 the user may take a call on accepting or declining the alert based on one's requirement. It is enquired at step 716 whether the current time is same as the scheduled time for consumption of the prescribed medication. If the answer to enquiry to step 716 is no, then the medication dispensing device clock continuously runs at step 718. If the answer to enquiry to step 716 is yes, then alerts are sent to the computing device and these alerts are reflected in the form of text messages and/or in the form of LED flashlights at step 720. These alerts are may be communicated to extended members whom the user intends to share the information with. Authentication by the patient by one of the acceptable methods for opening the medication dispensing device at step 722. The acceptable methods may not be limited to a local process and/or a remote process. These processes may not be further limited to: biometric measurements (for example, fingerprint verification, hand geometry, voice recognition, retinal scanning, signature verification, iris scanning, face recognition); image processing using artificial language or machine learning; and pressure processing. At step 724 the first user (patient) has an access to the medication and opening of the medication dispensing device post activation of the touch sensors. The opened medication dispensing device finally reverts to its position post the deactivation of the touch sensor at step 726.

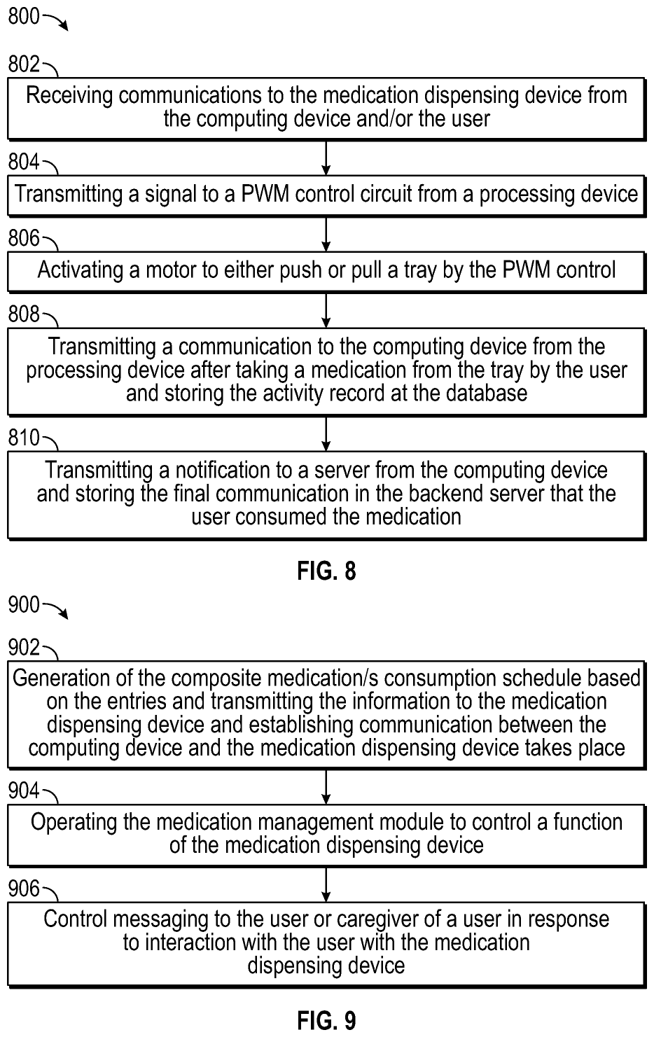

Referring to FIG. 8 is a flowchart 800, depicting the method for performing actions between the medication dispensing device and the computing device, according to an exemplary embodiment of the present disclosure. As an option, the method 800 may be carried out in the context of the details of FIG. 1, FIG. 2, FIG. 3, FIG. 4, FIG. 5, FIG. 6, and FIG. 7. However, the method 800 may also be carried out in any desired environment. Further, the aforementioned definitions may equally apply to the description below.

The method commences at step 802 where the communications are received to the processing device (medication dispensing device) from the computing device and/or the user. The communications, which may not be limited to, messages, user's biometric measurements, and the like. The processing device transmits a signal to the PWM control circuit at step 804. At step 806 the motor is activated to either push or pull the tray by the PWM control circuit. The processing device transmits the communication to the computing device and the store the activity at the database, the communication i.e., a medication is taken by the user from the tray at step 808. The user may take one or more medications from the tray in accordance with generated composite medications consumption schedule. At step 810 the computing device transmits the notification to the server and the backend server stores the final communication that the user consumed the medications.

Referring to FIG. 9 is a flowchart 900, depicting the method for managing actions by the medication management module, according to an exemplary embodiment of the present disclosure. As an option, the method 900 may be carried out in the context of the details of FIG. 1, FIG. 2, FIG. 3, FIG. 4, FIG. 5, FIG. 6, FIG. 7, and FIG. 8. However, the method 900 may also be carried out in any desired environment. Further, the aforementioned definitions may equally apply to the description below.

The method commences at step 902 by generation of the composite medication/s consumption schedule based on the entries and transmitting the information to the medication dispensing device and establishing communication between the computing device and the medication dispensing device. The medication management module may operate to control a function of the medication dispensing device at step 904. For example, the medication management module may operate the authentication features of the medication dispensing device. The medication management module may control messaging to the user or caregiver of a user in response to interaction with the user with the medication dispensing device at step 906. For example, if the medication management module determines that the user has not accessed medication held in the medication dispensing device according to the generated medication/s consumption schedule, the medication dispensing device may generate and deliver a message to the user or caregiver of the user as a reminder to access the medication via the medication dispensing device.

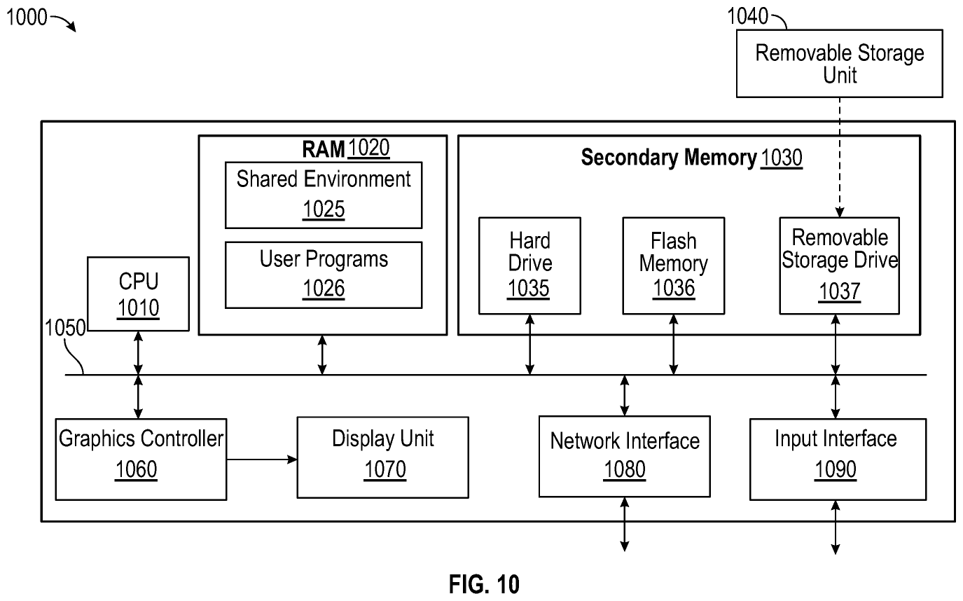

Referring to FIG. 10 is a block diagram 1000, depicting the details of the computing device 1000 in which various aspects of the present disclosure are operative by execution of appropriate software instructions. The device may correspond to computing devices such as the computing device 102a or 102b (or any other system in which the various features disclosed above can be implemented).

The end-user computing device 1000 may contain one or more processors such as a central processing unit (CPU) 1010, random access memory (RAM) 1020, secondary memory 1027, graphics controller 1060, display unit 1070, network interface 1080, and input interface 1090. All the components except display unit 1070 may communicate with each other over communication path 1050, which may contain several buses as is well known in the relevant arts. The components of FIG. 10 are described below in further detail.

CPU 1010 may execute instructions stored in RAM 1020 to provide several features of the present disclosure. CPU 1010 may contain multiple processing units, with each processing unit potentially being designed for a specific task. Alternatively, CPU 1010 may contain only a single general-purpose processing unit.

RAM 1020 may receive instructions from secondary memory 1030 using communication path 1050. RAM 1020 is shown currently containing software instructions, and/or data such as those used in threads and stacks, constituting shared environment 1025 and/or user programs 1026. Shared environment 1025 includes operating systems, device drivers, virtual machines, etc., which provide a (common) run time environment for execution of user programs 1026. The RAM 1020 may also be configured to store the modules of the medication management module 112.

Graphics controller 1060 generates display signals (e.g., in RGB format) to display unit 1070 based on data/instructions received from CPU 1010. Display unit 1070 contains a display screen to display the images defined by the display signals. Input interface 1090 may correspond to a keyboard and a pointing device (e.g., touch-pad, mouse) and may be used to provide inputs. The network interface 1080 provides connectivity to a network (e.g., using Internet Protocol), and may be used to communicate with other systems (such as those shown in FIG. 1, network 106 connected to the network.

Secondary memory 1030 may contain hard drive 1035, flash memory 1036, and removable storage drive 1037. Secondary memory 1030 may store the data software instructions (e.g., for performing the actions noted above with respect to the Figures), which enable digital processing system 1000 to provide several features in accordance with the present disclosure. The flash memory 1036 may include an electronic non-volatile computer storage medium that can be electrically erased and reprogrammed.

Some or all of the data and instructions may be provided on removable storage unit 1040, and the data and instructions may be read and provided by removable storage drive 1037 to CPU 1010. Floppy drive, magnetic tape drive, CD-ROM drive, DVD Drive, Flash memory, removable memory chip (PCMCIA Card, EEPROM) are examples of such removable storage drive 1037.

Removable storage unit 1040 may be implemented using medium and storage format compatible with removable storage drive 1037 such that removable storage drive 1037 can read the data and instructions. Thus, removable storage unit 1040 includes a computer readable (storage) medium having stored therein computer software and/or data. However, the computer (or machine, in general) readable medium can be in other forms (e.g., non-removable, random access, etc.).

In this document, the term “computer program product” is used to generally refer to removable storage unit 1040 or hard disk installed in hard drive 1035. These computer program products are means for providing software to digital processing system 1000. CPU 1010 may retrieve the software instructions, and execute the instructions to provide various features of the present disclosure described above.

The term “storage media/medium” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage memory 1030. Volatile media includes dynamic memory, such as RAM 1020. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 1050. Transmission media can also take the form of acoustic or light waves, such as those generated during radio-wave and infra-red data communications.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A smart system for dispensing medications, comprising:
a plurality of computing devices in wireless communication with at least one medication dispensing device, the plurality of computing devices comprises of a medication management module configured to generate schedules and timetables based on a plurality of inputs provided by a plurality of users with reference to the medication intended to be housed in the at least one medication dispensing device based on the consumption requirement, wherein the medication management module configured to send the generated schedules and timetables to the at least one medication dispensing device; and
cloud servers are configured to establish communication with the plurality of computing devices and the at least one medication dispensing device via a network, the cloud servers are configured to store the conversations between the plurality of computing devices and the at least one medication dispensing device.

2. The smart system of claim 1, wherein the at least one medication dispensing device comprises at least one controlling and communication unit connected to at least one motor.

3. The smart system of claim 1, wherein the at least one medication device further comprises a plurality of trays with a plurality of compartments having dosage of medications.

4. The smart system of claim 3, wherein the plurality of trays with the plurality of compartments opens and closes with the aid of the at least one motor.

5. The smart system of claim 2, wherein the at least one controlling and communication unit comprises a processing device connected to at least one pulse width modulation (PWM) control circuit, at least one real time clock circuit, at least one first antenna, at least one second antenna, and at least one power source.

6. The smart system of claim 1, wherein the at least one medication dispensing device receives communications from the plurality of computing devices, a plurality of signals transmitted to the PWM control circuit from the at least one processing device, thus activating the at least one motor to either push or pull the plurality of trays with the plurality of compartments by the at least one PWM control circuit.

7. The smart system of claim 5, wherein the real time clock circuit is configured to generate the schedule for each unit of the prescribed medication in each tray of the at least one medication dispensing device.

8. The smart system of claim 5, wherein the at least one first antenna and the at least one second antenna are configured to establish communication with the plurality of computing devices by the dedicated wireless communication technologies via the network.

9. The smart system of claim 1, wherein the at least one medication dispensing device comprises at least one speaker configured to alert at least one user when it is time to take a medication or when the medication has not been take.

10. The smart system of claim 1, wherein the at least one medication dispensing device comprises at least one first motor configured to shift at least one first robotic arm from left to right and vice versa.

11. The smart system of claim 1, wherein the at least one medication dispensing device comprises at least one second motor configured to shift at least one second robotic arm from up to down and vice versa.

12. The smart system of claim 1, wherein the at least one medication dispensing device comprises at least one third motor configured to shift at least one third robotic arm from up to down and vice versa.

13. The smart system of claim 10, 11, 12, wherein the at least one first robotic arm, the at least one second robotic arm, and the at least one third robotic arm are configured to push or pull the plurality of trays.

14. The system of claim 1, wherein the at least one medication dispensing device comprises at least one circuitry connected to the at least one first motor, the at least one second motor, and the at least one third motor.

15. The smart system of claim 1, wherein the at least one medication dispensing device comprises at least one first antenna and at least one second antenna configured to establish communication with the plurality of computing devices via the network.

16. The smart system of claim 1, wherein the at least one medication dispensing device configured to send the notifications to the servers and the plurality of computing devices after taking the medications from the at least one medication dispensing device by the user.

17. The smart system of claim 1, further comprising a database configured to generate a plurality of real time alerts to the plurality of computing devices.

18. A method for dispensing time bound medications, comprising:
logging into a medication management module associated with a plurality of computing devices;
generating composite medications consumption schedules and timetables based on a plurality of inputs provided by a plurality of users with reference to the medication intended to be housed in a medication dispensing device based on the consumption requirement;
transmitting the information to the medication dispensing device and establishing communication between the plurality of computing devices and the medication dispensing device;
suggesting the changes to the plurality of users depending upon the entry relevant to the dose of medication in the form of a database alert;
sending alerts as a LED flash light and/or text messages to the plurality of computing devices; and
accessing the medication by the plurality of users and opening of the medication dispensing device post activation of the touch sensors.

19. A computer program product comprising a non-transitory computer-readable medium having a computer-readable program code embodied therein to be executed by one or more processors, the program code including instructions to:
log into a medication management module associated with a plurality of computing devices;
generate composite medications consumption schedules and timetables based on a plurality of inputs provided by a plurality of users with reference to the medication intended to be housed in a medication dispensing device based on the consumption requirement;
transmit the information to the medication dispensing device and establishing communication between the plurality of computing devices and the medication dispensing device;
suggest the changes to the plurality of users depending upon the entry relevant to the dose of medication in the form of a database alert;
send alerts as a LED flash light and/or text messages to the plurality of computing devices; and
access the medication by the plurality of users and opening of the medication dispensing device post activation of the touch sensors.
